(19) United States
(12) Patent Application Publication
(10) Pub. No.: US 2011/0184024 A1
(43) Pub. Date: Jul. 28, 2011

(54) Pharmaceutical Composition Containing Esomeprazole

(75) Inventors: Seong-Shin Kwak, Seoul (KR); Bong-Sang Lee, Gyeonggi-do (KR); Do-Woo Kwon, Chungcheongnam-do (KR); Hong Ryeol Jeon, Gyeonggi-do (KR); Ji-Yun Moon, Gyeonggi-do (KR); Dong-Jin Lee, Gyeonggi-do (KR)

(73) Assignee: CTC BIO, INC., Seoul (KR)

(21) Appl. No.: 12/680,236

(22) PCT Filed: Sep. 16, 2008
(86) PCT No.: PCT/KR2008/005451

§ 371 Date: Mar. 26, 2010

(30) Foreign Application Priority Data
Sep. 28, 2007 (KR) 10-2007-0098095

(51) Int. Cl. A61K 31/4439; A61P 1/04

(52) U.S. Cl. 514/338

(57) ABSTRACT

The present invention relates to esomeprazole free base or its alkali salt-containing composition which stability is improved and is easy to manufacture.

### TECHNICAL FIELD

The present invention relates to esomeprazole free base or its alkali salt-containing pharmaceutical preparation which stability is improved.

### BACKGROUND ART

Esomeprazole ((S)-5-methoxy-2-[(4-methoxy-3,5-dimethylpyridin-2-yl)methylsulfinyl]-3H-benzoimidazole) is a proton pump inhibitor which is used in the treatment of peptic ulcer, gastroesophageal reflux etc. The free base of the esomeprazole is not good in terms of storage stability and physical properties. Concretely, esomeprazole free base's melting point is about 45° C. so that it is much difficult to formulate, and its stability is so bad that total impurities increase to about 1% after stored for two months in conditions of 25° C./60% RH. In addition, the esomeprazole's stability is much worse in alkali condition than acidic or neutral condition.

A commercially available product uses esomeprazole magnesium salt to improve stability and physical properties of the esomeprazole free base. However, the magnesium salt of esomeprazole also does not have enough good stability, and thus many studies have been performed to improve the stability of the salt.

### DISCLOSURE

### Technical Problem

Accordingly, the object of the present invention is to provide an esomeprazole free base or its alkali salt-containing pharmaceutical composition which stability is improved and is simple to manufacture.

### Technical Solution

To achieve the object, the present invention provides a solid dispersion comprising esomeprazole free base or its alkali salt; alkaline material; and hydrophilic polymer selected from the group consisting of polyvinylpyrrolidone, vinylpyrrolidone-vinylacetate copolymer and their mixture.

Preferably, the present invention provides said solid dispersion wherein the alkaline material is MgO or Mg(OH)₂.

The prevent invention provides also an esomeprazole-containing pharmaceutical composition having improved stability, which comprises said solid dispersion.

Hereinafter, the esomeprazole-containing solid dispersion and the pharmaceutical composition comprising the solid dispersion according to the present invention will be described in more detail.

The present invention provides a solid dispersion comprising esomeprazole free base or its alkali salt; alkaline material; and hydrophilic polymer selected from the group consisting of polyvinylpyrrolidone, vinylpyrrolidone-vinylacetate copolymer and their mixture.

The present invention is based on the fact that the stability of the solid dispersion made of esomeprazole free base or its alkali salt, alkaline material and hydrophilic polymer is much better than the simple mixture of esomeprazole free base or its alkali salt, alkaline material and hydrophilic polymer. The present invention is based on also the surprising fact that polyvinylpyrrolidone, vinylpyrrolidone-vinylacetate copolymer or their mixture is very much better for esomeprazole's stability in the solid dispersion than other polymers.

The solid dispersion according to the present invention comprises esomeprazole free base or its alkali salt as active agent, and alkali metal salt or alkali earth metal salt such as esomeprazole's sodium, calcium, potassium, magnesium, manganese, iron, copper, zinc, aluminum or lithium salt and esomeprazole salt made from basic amino acid such as arginine, lysine or glycine, ammonia, the first, second or tertiary amine and so on may be used as the esomeprazole's alkali salt.

The solid dispersion according to the present invention comprises also polyvinylpyrrolidone, vinylpyrrolidone-vinylacetate copolymer or their mixture as hydrophilic polymer, which forms and keeps solid dispersion state. The polyvinylpyrrolidone and vinylpyrrolidone-vinylacetate copolymer are much better in improving the stability of esomeprazole than other hydrophilic polymers.

Further, the solid dispersion according to the present invention comprises alkaline material to make the micro-environment of the inside of the solid dispersion alkaline for the purpose of improving the stability of esomeprazole. Examples of the alkaline material include, but are not limited to, inorganic alkaline material like hydroxide, oxide, carbonate or phosphate made from alkali metal or alkali earth metal such as sodium, calcium, potassium, magnesium, manganese, iron, copper, zinc, aluminum or lithium; and organic alkaline material like basic amino acid such as arginine, lysine or glycine, ammonia, the first, second or tertiary amine, cyclic amine, meglumine (N-methyl-D-glutamine). Among these alkaline materials, magnesium oxide and magnesium hydroxide are much better in terms of esomeprazole's stability than other alkaline materials.

Therefore, most preferably, the present invention provides the solid dispersion comprising esomeprazole free base or its alkali salt; polyvinylpyrrolidone, vinylpyrrolidone-vinylacetate copolymer or their mixture; and magnesium oxide or magnesium hydroxide.

In case that polyvinylpyrrolidone or vinylpyrrolidone-vinylacetate copolymer and magnesium oxide or hydroxide are added to improve the stability of esomeprazole, it is preferable that polyvinylpyrrolidone or vinylpyrrolidone-vinylacetate copolymer; magnesium oxide or hydroxide; and esomeprazole are mixed homogenously and minutely. In this case, making solid dispersion can very much improve the stability of esomeprazole.

This solid dispersion can be made by dissolving or suspending esomeprazole free base or its alkali salt; polyvinylpyrrolidone, vinylpyrrolidone-vinylacetate copolymer or their mixture; and magnesium oxide or hydroxide together in a suitable solvent like water, ethanol, isopropanol, acetone, methanol, or methylene chloride, and then drying the solution or suspension in a condition to keep the mixture's homogeneity.

The solid dispersion according to the present invention may be made by drying the solution or suspension only, or by spray-drying the solution or suspension onto a pharmaceutically acceptable other excipient (for example, lactose, microcrystalline cellulose, sucrose, starch etc.).

Besides said methods, the solid dispersion according to the present invention may be made by making a solid dispersion comprising esomeprazole free base or its alkali salt and polyvinylpyrrolidone, vinylpyrrolidone-vinylacetate copolymer or their mixture, and then spray-drying magnesium oxide or hydroxide onto the solid dispersion; or by making a solid dispersion comprising esomeprazole free base or its alkali salt and magnesium oxide or hydroxide, and then spray-drying polyvinylpyrrolidone, vinylpyrrolidone-vinylacetate copolymer or their mixture onto the solid dispersion. However, the method to dissolve or suspend three components together for making a solid dispersion is most preferable in terms of homogeneity of mixture, and, consequently, in terms of stability.

Therefore, preferably, the present invention provides also a method for manufacturing esomeprazole-containing solid dispersion which has improved stability, wherein the method comprises (S1) preparing a solution or suspension comprising esomeprazole free base or its alkali salt; magnesium oxide or hydroxide; and polyvinylpyrrolidone, vinylpyrrolidone-vinylacetate copolymer or their mixture; and (S2) drying the solution or suspension.

More preferably, the present invention provides said method for manufacturing esomeprazole-containing solid dispersion which has improved stability, wherein the LOD (loss on drying, 105° C., the weight of test sample is 5g) in the drying process of the (S2) step is less than 2% (0.5-2%), more preferably less than 1.5% (0.5-1.5%).

Preferably, in the method, a small amount of alkaline material may be further added into the solution or suspension to block esomeprazole from degrading during the process for making the solid dispersion.

The present invention provides also a pharmaceutical composition comprising the solid dispersion, and the pharmaceutical composition may comprise pharmaceutically acceptable excipients in addition to the solid dispersion. Examples of the pharmaceutically acceptable excipient include, but are not limited to, diluent, favoring agent, binder, preservative, disintegrator, lubricant, and filler.

The present invention provides also a pharmaceutical preparation which is made by coating said pharmaceutical composition with enteric polymer (for example, methacrylic acid copolymer dispersion, hydroxypropylmethylcellulose phthalate, hydroxypropylmethylcellulose acetate succinate, cellulose acetate phthalate, or polyvinyl acetate phthalate), and, more preferably, said pharmaceutical preparation which has another coating layer made of hydrophilic polymer (hydroxypropylcellulose, hydroxypropylmethylcellulose, polyvinylpyrrolidone, vinylpyrrolidone-vinylacetate copolymer, polyvinylalcohol, polyvinylalcohol-polyethylene glycol copolymer, hydroxymethylcellulose, hydroxyethylcellulose, hydroxyethylmethylcellulose, polyvinylacetate, polyalkene oxide, polyalkene glycol, polyoxyethylene-polyoxypropylene polymer, diethylamino acetate, aminoalkylmethacrylate copolymer, sodium alginate, gelatin etc.) between the pharmaceutical composition and the enteric coating layer.

### Advantageous Effects

The present invention provides an esomeprazole free base or its alkali salt-containing pharmaceutical composition which has improved stability and is easy to manufacture.

### BRIEF DESCRIPTION OF THE DRAWINGS

FIG. 1 is graphs showing pharmacokinetic absorption evaluation results performed before a meal with a preferable example according to the present invention and Nexium 40 mg, comparative example.

FIG. 2 is graphs showing pharmacokinetic absorption evaluation results performed after a meal with a preferable example according to the present invention and Nexium 40 mg, comparative example.

### MODES FOR CARRYING OUT THE INVENTION

Hereinafter, the present invention is described in considerable detail to help those skilled in the art understand the present invention. However, the following examples are offered by way of illustration and are not intended to limit the scope of the invention. It is apparent that various changes may be made without departing from the spirit and scope of the invention or sacrificing all of its material advantages.

### Experimental Example 1

### Selection of Polymer

As shown in table 1 below, each polymer was mixed with esomeprazole free base at a weight ratio of 1:3 or 1:6, and the each mixture was compacted with roller compacter and granulated. All conditions were the same to counterbalance temperature happening during compaction. Stability test was performed by storing made granules in brown plastic bottles at accelerated conditions 75% RH) for one week, and formed impurities were analyzed with HPLC. The results were collectively shown in tables 2 and 3.

The analysis of impurities was performed as follows: each granule was pulverized, and an amount equivalent to about 20 mg of esomeprazole was weighed and put in 100 mL of flask. 10 mL of methanol was added in the flask, and mixed well. Then 20 mL of phosphate buffer solution (pH 11.0) was added, and the flask was sonicated. Water was added to make the same volume, and then the solution was membrane-filtered to make a test sample. Then, the test sample was analyzed in HPLC conditions described below. Peak area of less than 0.05% was not included in the calculation.

[HPLC Conditions]

Detector: UV spectrophotometer (detection wavelength 302 nm)

Column: Cosmosil 5C18-AR-II, 4.6×150 mm or its equivalent column

Mobile phase: (Gradient)

A: a mixture of acetonitrile, phosphate buffer solution (pH 7.6) and water (10/10/80)

B: a mixture of acetonitrile, phosphate buffer solution (pH 7.3) and water (80/1/19)

Injection volume: 20 uL

Flow rate: 1.0 mL/min

TABLE 1

| | Mixing ratio 1:3 | | | | Mixing ratio 1:6 | | | |
|---|---|---|---|---|---|---|---|---|
| Ingredient | 1 | 2 | 3 | 4 | 5 | 6 | 7 | 8 |
| Esomeprazole | 40 | 40 | 40 | 40 | 40 | 40 | 40 | 40 |
| HPMC (5 cp) | 120 | — | — | — | 240 | — | — | — |
| HPMC (15 cp) | — | 120 | — | — | — | 240 | — | — |
| L-HPC | — | — | 120 | — | — | — | 240 | — |
| HPC | — | — | — | 120 | — | — | — | 240 |

In the table 1, HPMC 5 cp and 15 cp mean hydroxypropylmethylcellulose which viscosity is about 5 cP or 15 cP, respectively, when measured at 20° C. as 2 wt % water solution. L-HPC means low-substituted hydroxypropylcellulose, and HPC means hydroxypropylcellulose.

TABLE 2

| | | Single max. | | Total | |
|---|---|---|---|---|---|
| | | impurity | | impurities | |
| | Mixing | | After | | After |
| Ingredient | ratio (w/w) | Initial | 1 week | Initial | 1 week |
| HPMC 5cp | 1:3 | 0.08 | 0.51 | 0.32 | 3.66 |
| HPMC 15cp | 1:3 | 0.10 | 1.62 | 0.83 | 13.48 |
| L-HPC | 1:3 | 0.08 | 0.96 | 0.48 | 6.39 |
| HPC | 1:3 | 0.12 | 1.16 | 0.56 | 7.51 |

TABLE 3

| | | Single max. | | Total | |
|---|---|---|---|---|---|
| | | impurity | | impurities | |
| | Mixing | | After | | After |
| Ingredient | ratio (w/w) | Initial | 1 week | Initial | 1 week |
| HPMC 5cp | 1:6 | 0.09 | 0.62 | 0.30 | 2.68 |
| HPMC 15cp | 1:6 | 0.13 | 1.64 | 0.82 | 13.89 |
| L-HPC | 1:6 | 0.08 | 0.77 | 0.45 | 4.53 |
| HPC | 1:6 | 0.16 | 1.24 | 0.58 | 7.84 |

As shown in the tables 1 and 2, the compacted mixture using HPMC 5 cp was the most stable in case of forming compacted mixture of esomeprazole free base and hydrophilic polymer for solid dispersion.

### Experimental Example 2

### Manufacture and Evaluation of Solid Dispersion

Solid dispersions were made according to ingredients and contents of table 4 below. Example 1 was made as follows: polyvinylpyrrolidone was dissolved in ethanol, to which NaOH water solution was added and then mixed. Esomeprazole free base was dissolved in the mixed solution. Then lactose and colloidal silicon dioxide were added to the solution and mixed. The solution was dried at 40° C. and granulated to make solid dispersion granules. Example 2 was made as follows: polyvinylpyrrolidone was dissolved in ethanol, to which esomeprazole free base was dissolved. Then magnesium oxide and colloidal silicon dioxide were added to the solution and mixed. The solution was dried at 40° C. and granulated to make solid dispersion granules. Example 3 was made as follows: hydroxypropylmethylcellulose (5 cP) was dissolved in ethanol, to which NaOH water solution was added. Esomeprazole free base was dissolved in the solution. Then magnesium oxide and colloidal silicon dioxide were added to the solution and mixed. The solution was dried at 40° C. and granulated to make solid dispersion granules.

TABLE 4

| | Amount (mg) | | |
|---|---|---|---|
| Ingredient | Example 1 | Example 2 | Example 3 |
| Esomeprazole | 40 | 40 | 40 |
| NaOH | 1.8 | — | 1.8 |
| PVP | 40 | 40 | — |
| HPMC 5 cP | — | — | 40 |
| MgO | — | 80 | 80 |
| Lactose | 80 | — | — |
| Colloidal SiO₂ | 30 | 30 | 30 |
| Total | 191.8 | 190.0 | 191.8 |

The stability of examples 1 to 3 was evaluated according to the same method as that of experimental example 1. Results were shown in table 5 below.

TABLE 5

| | Initial | | 1 week | | 2 weeks | |
|---|---|---|---|---|---|---|
| | Single | | Single | | Single | |
| Sample ID | max. | Total | max. | Total | max. | Total |
| Example 1 | 0.088 | 0.483 | 1.249 | 3.556 | — | — |
| Example 2 | 0.072 | 0.350 | 0.125 | 0.787 | 0.205 | 0.892 |
| Example 3 | 0.062 | 0.290 | 0.277 | 1.034 | 0.637 | 2.224 |

As shown in the table 5, the solid dispersion of example 1 having no MgO showed much increase of impurities after only 1 week of the accelerated stability test. In addition, the stability of the solid dispersion using PVP was much better than the solid dispersion using HPMC 5 cP which showed the best stability in experimental example 1

### Experimental Example 3

### Stability Test of Simple Mixture

Simple mixture, a test sample was made by simply mixing according to the ingredients and contents of table 6.

TABLE 6

| | Amount (mg) | | |
|---|---|---|---|
| Ingredient | Example 4 | Example 5 | Example 6 |
| Esomeprazole | 40 | 40 | 40 |
| PVP | 40 | — | 40 |
| MgO | 80 | 80 | — |

The stability of examples 4 to 6 was evaluated according to the same method as that of experimental example 1. Results were shown in table 7 below.

TABLE 7

| | Initial | | 1 week | |
|---|---|---|---|---|
| Sample ID | Single max. | Total | Single max. | Total |
| Example 4 | 0.051 | 0.234 | 1.181 | 9.503 |
| (Active agent + PVP + MgO) | | | | |
| Eexample 5 | 0.053 | 0.231 | 0.892 | 6.568 |
| (Active agent + MgO) | | | | |

| | | | |
|---|---|---|---|
| Example 6 | 0.050 | 0.241 | Browing |
| (Active agent + PVP) | | | (Dark brown) |

As shown in the table 7, simple mixture of ingredients did not show improved stability of esomeprazole.

### Experimental Example 4

### Evaluation of Alkaline Material

As shown in table 8 below, the effect of the kind of alkaline material on stability was evaluated in esomeprazole-containing solid dispersion according to the present invention. PVP was dissolved in ethanol, to which esomeprazole free base was dissolved. Each alkaline material and colloidal silicon dioxide were added to the solution and mixed well. The solution was dried at 40° C., and then granulated to make solid dispersion granules.

TABLE 8

| | | Example | | | | | | |
|---|---|---|---|---|---|---|---|---|
| Ingredient | | 7 | 8 | 9 | 10 | 11 | 12 | 13 |
| Esomeprazole | | 40 | 40 | 40 | 40 | 40 | 40 | 40 |
| PVP | | 40 | 40 | 40 | 40 | 40 | 40 | 40 |
| alkaline | MgO | 80 | — | — | — | — | — | — |
| material | | | | | | | | |
| alkaline | MgCO₃ | — | 80 | — | — | — | — | — |
| material | | | | | | | | |
| alkaline | Na₂CO₃ | — | — | 80 | — | — | — | — |
| material | | | | | | | | |
| alkaline | NaHCO₃ | — | — | — | 80 | — | — | — |
| material | | | | | | | | |
| alkaline | K₂HPO₄ | — | — | — | — | 80 | — | — |
| material | | | | | | | | |
| alkaline | CaCO₃ | — | — | — | — | — | 80 | — |
| material | | | | | | | | |
| alkaline | Na₂HPO₄ | — | — | — | — | — | — | 80 |
| material | | | | | | | | |

| | | | | | | | |
|---|---|---|---|---|---|---|---|
| Colloidal SiO₂ | 30 | 30 | 30 | 30 | 30 | 30 | 30 |

The stability of examples 7 to 13 was evaluated according to the same method as that of experimental example 1. Results were shown in table 9 below.

TABLE 9

| | alkaline | Initial | | 1 week | |
|---|---|---|---|---|---|
| Sample ID | material | Single max. | Total | Single max. | Total |
| Example 7 | MgO | 0.082 | 0.295 | 0.105 | 0.521 |
| Example 8 | MgCO₃ | 0.088 | 0.281 | 0.486 | 1.919 |
| Example 9 | Na₂CO₃ | 0.062 | 0.307 | 0.236 | 0.894 |
| Example 10 | NaHCO₃ | 0.111 | 0.447 | 0.852 | 2.589 |
| Example 11 | K₂HPO₄ | 0.087 | 0.399 | 0.545 | 1.786 |
| Example 12 | CaCO₃ | 0.153 | 0.568 | Browning | Dark |
| | | | | | brown |
| Example 13 | Na₂HPO₄ | 0.200 | 0.755 | Browning | Dark |
| | | | | | brown |

As shown in table 9, the solid dispersion using MgO was the most stable.

### Experimental Example 5

### Evaluation of Content of Each Ingredient

Esomeprazole free base-containing solid dispersion was made according to the ingredients and contents of table 10 below. Polyvinylpyrrolidone was dissolved in ethanol, to which NaOH water solution was added. Esomeprazole free base was added to the mixed solution, and then MgO and colloidal silicon dioxide were added and mixed. The solution was dried at 40° C. and granulated to make solid dispersion granules.

TABLE 10

| | Amount (mg) | | | | |
|---|---|---|---|---|---|
| | Example | Example | Example | Example | Exam- |
| Ingredient | 14 | 15 | 16 | 17 | ple 18 |
| Esomeprazole | 40 | 40 | 40 | 40 | 40 |
| NaOH | 2 | 2 | 5 | 2 | 2 |
| PVP | 40 | 40 | 40 | 20 | 40 |
| MgO | 80 | 100 | 100 | 100 | 100 |
| Colloidal SiO₂ | 30 | 30 | 30 | 30 | 0 |

The stability of examples 14 to 18 was evaluated according to the same method as that of experimental example 1. Results were shown in table 11 below.

TABLE 11

| | |
|---|---|
| Initial | 11 days |

| Sample ID | Single max. | Total | Single max. | Total |
|---|---|---|---|---|
| Example 14 | 0.098 | 0.353 | 0.148 | 0.540 |
| Example 15 | 0.112 | 0.270 | 0.112 | 0.425 |
| Example 16 | 0.096 | 0.514 | 0.162 | 0.574 |
| Example 17 | 0.096 | 0.260 | 0.154 | 0.825 |
| Example 18 | 0.110 | 0.320 | 0.121 | 0.529 |

As shown in the table 11, colloidal silicon dioxide is thought not to affect the stability of esomeprazole when comparing example 15 with example 18. In addition, the more the amount of MgO was, the better the stability of esomeprazole-containing solid dispersion was, when comparing example 14 and example 15, and the decrease of PVP content caused the increase of total impurities when comparing example 15 and example 17.

### Experimental Example 6

### Stability Evaluation According to Water Content in Process Steps

Esomeprazole free base-containing pharmaceutical composition was made according to the table 12 below, and only drying process was changed in examples a to e to evaluate the affect of drying process on stability.

TABLE 12

| No. | Ingredient | Amount (mg) |
|---|---|---|
| 1 | Esomeprazole | 40.0 |
| 2 | Povidone | 60.0 |
| 3 | Magnesium oxide | 60.0 |
| 4 | Spherical sugar | 138.0 |
| 5 | Hydroxypropylmethylcellulose | 20.0 |
| 6 | Methacrylic acid copolymer dispersion | 238.3 |
| 7 | Polyethylene glycol | 4.0 |
| 8 | Citric acid triethyl | 14.3 |
| 9 | Talc | 13.6 |
| 10 | Colloidal silicon dioxide | 1.0 |
| 11 | Titanium oxide | 3.6 |

| | |
|---|---|
| Total | 426.0 |

Examples a to e were made as follows: povidone was dissolved in ethanol, to which magnesium oxide was suspended and then esomeprazole was dissolved. The solution was coated onto spherical sugars by using a fluid bed coater to make solid dispersion granules. Then the granules were dried (drying after the first coating). The granules were again coated with HPMC to make a separating layer and then dried (drying after the second coating). Then the granules were again coated with methacrylic acid copolymer dispersion to make an enteric coating layer, and dried (drying after the third coating) to make esomeprazole free base-containing granules. LOD (Moisture Balance (Precisa, XM60), drying temperature 105° C., Auto stop, sample weight 5 g) measured after each drying step were shown in table 13 below.

TABLE 13

| Batch no. | Example a | Example b | Example c | Example d | Example e |
|---|---|---|---|---|---|
| Drying after | * | * | * | 1.93 | 1.27 |
| 1^{st} coating | | | | | |
| Drying after | 2.38 | 2.09 | 1.23 | 1.76 | 1.30 |
| 2^{nd} coating | | | | | |
| Drying after | 1.97 | 1.75 | 1.51 | 1.12 | 1.29 |
| 3^{rd} coating | | | | | |

*: The 2^{nd} coating was performed continuously after 1^{st} coating, without drying step after 1^{st} coating.

Results of stability test using the examples a to e were shown in tables 14 and 15 below. Table 14 shows results of samples packed with PTP alu-alu, stored for 1 week at harsh storage condition (60° C., 75% RH), and table 15 shows results of samples packed with PTP alu-alu, stored for 6 months at accelerated storage condition (40° C., 75% RH).

TABLE 14

| | |
|---|---|
| | 1 week |
| initial | (harsh condition) |

| Batch no. | Single max. | Total | Single max. | Total |
|---|---|---|---|---|
| Example a | 0.041 | 0.146 | 6.40 | 18.33 |
| Example b | 0.025 | 0.109 | 2.40 | 10.91 |
| Example c | 0.046 | 0.209 | 0.85 | 2.90 |
| Example d | 0.043 | 0.207 | 0.18 | 0.53 |
| Example e | 0.047 | 0.224 | 0.15 | 0.50 |

TABLE 15

| | Initial | | 3 months | | 6 months | |
|---|---|---|---|---|---|---|
| | Single | | Single | | Single | |
| Batch no. | max. | Total | max. | Total | max. | Total |
| Example b | 0.025 | 0.109 | 0.335 | 1.762 | — | — |
| Example c | 0.046 | 0.209 | 0.148 | 1.364 | 0.216 | 2.135 |
| Example e | 0.047 | 0.224 | 0.075 | 0.492 | 0.127 | 1.139 |

As shown in the tables 14 and 15, examples d and e showed greatly improved stability compared to other batch not controlling water content of coated granules in each process. Particularly, controlling water content of the first drying step among three drying processes severely affected stability, even if water content control in all coating processes were important. Drying after the first coating is preferably performed until LOD is less than 2%, more preferably less than 1.5%.

### Experimental Example 7

### Evaluation of Pharmacokinetic Absorption

Enteric-coated tablets, test samples were made according to tables 16 and 17 to evaluate pharmacokinetic absorption of pharmaceutical preparations of the present invention. Concretely, povidone was dissolved in ethanol, to which NaOH water solution was added. Then esomeprazole was dissolved in the solution and mixed well. Some portion of magnesium oxide was suspended in the solution. The solution was sprayed onto colloidal silicon dioxide and magnesium oxide in fluid bed to make granules. The granules were mixed with other ingredients and tabletted to make a core tablet before coating. Then, the core tablets were coated with HPMC to make a separating layer by a tablet coater. The coated tablet was again enteric-coated with HPMC P(HP-50) to make example A, and with methacrylic acid ethylacrylate copolymer (1:1) to make example B.

TABLE 16

| No. | Ingredient (core tablet) | Core tablet |
|---|---|---|
| 1 | Esomeprazole | 40 |
| 2 | Povidone | 40 |
| 3 | NaOH | 1.8 |
| 4 | Magnesium oxide | 100 |
| 5 | Colloidal SiO₂ | 12.5 |
| 6 | Microcrystalline cellulose | 265.7 |
| 7 | Crospovidone | 25.0 |
| 8 | Magnesium stearate | 5.0 |

| | |
|---|---|
| Total | 490.0 |

TABLE 17

| | | Coating | |
|---|---|---|---|
| | | | Example |
| No. | Ingredient (coating formulation) | Example A | B |
| 1 | Core tablet | 490.0 | 490.0 |
| 2 | Hydroxypropylmethylcellulose | 9.8 | 17.2 |
| 3 | HPMC P(HP-50) | 50.23 | — |
| 4 | Methacrylic acid copolymer dispersion | — | 170.3 |
| 5 | PEG 6000 | 0.98 | 2.06 |
| 6 | TEC | — | 7.7 |
| 7 | PG | 4.02 | — |
| 8 | Talc | 8.97 | 8.85 |
| 9 | Titanium oxide | — | 5.1 |

| | | |
|---|---|---|
| Total | 564 | 582 |

Capsule preparation according to the present invention was made like table 18. The below two examples were made by the same method except for spherical sugar used as seed. 850-710 um of spherical sugar was used as seed for example C, and 710-600 um of spherical sugar was used as seed for example D. Povidone was dissolved in ethanol, to which magnesium oxide was suspended. Esomeprazole was dissolved in the solution. The solution was sprayed onto spherical sugar in a fluid bed to make pellets coated with active agent. These pellets were coated with HPMC to make a separating layer which blocks contact between active agent and enteric substance. Then the enteric-coated pellet was made with methacrylic acid copolymer dispersion.

TABLE 18

| | | | | Example |
|---|---|---|---|---|
| No. | Ingredient | | Example C | D |
| 1 | Esomeprazole | | 40.0 | 40.0 |
| 2 | Povidone | | 60.0 | 60.0 |
| 3 | Magnesium oxide | | 60.0 | 60.0 |
| 4 | Spherical | 710~600 um | — | 138.0 |
| | sugar | 850~710 um | 138.0 | — |

| | | | |
|---|---|---|---|
| 5 | HPMC | 20.0 | 20.0 |
| 6 | Methacrylic acid copolymer dispersion | 238.3 | 238.3 |
| 7 | Polyethylene glycol | 4.0 | 4.0 |
| 8 | Citric acid triethyl | 14.3 | 14.3 |
| 9 | Talc | 13.6 | 13.6 |
| 10 | Colloidal silicon dioxide | 1.0 | 1.0 |
| 11 | Titanium oxide | 3.6 | 3.6 |

| | | |
|---|---|---|
| Total | 426.0 | 426.0 |

Pharmacokinetic absorption test was performed with the examples A to D and commercially available product, Nexium 40 mg tablet (AstraZeneca, comparative example). Results of pharmacokinetic test performed before a meal were shown in table 19 (result of enteric-coated tablet) and 20 (result of capsule), respectively. Results of the tables 19 and 20 were mean value of values obtained after administering to 12 about 30-year-old men.

TABLE 19

| Batch no. | Example A | Example B | Comparative example |
|---|---|---|---|
| Cmax | 811.5 | 684.8 | 1141.8 |
| AUC | 3816.0 | 2947.6 | 3336.9 |
| Tmax | 4.3 | 3.8 | 2.3 |

In tablets, example A coated with HPMC P(HP-50) which dissolve at pH 5.0 showed higher Cmax and AUC than example B coated with methacrylic acid copolymer dispersion which dissolves at pH 5.5. When compared to the comparative example (commercially available product), example A showed 71.1% of Cmax and 114.4% of AUC, and example B showed 60.0% of Cmax and 88.3% of AUC.

TABLE 20

| Batch no. | Example C | Example D | Comparative example |
|---|---|---|---|
| Cmax | 1004.2 | 1237.5 | 1141.8 |
| AUC | 3556.6 | 3206.2 | 3336.9 |
| Tmax | 1.9 | 2.1 | 2.3 |

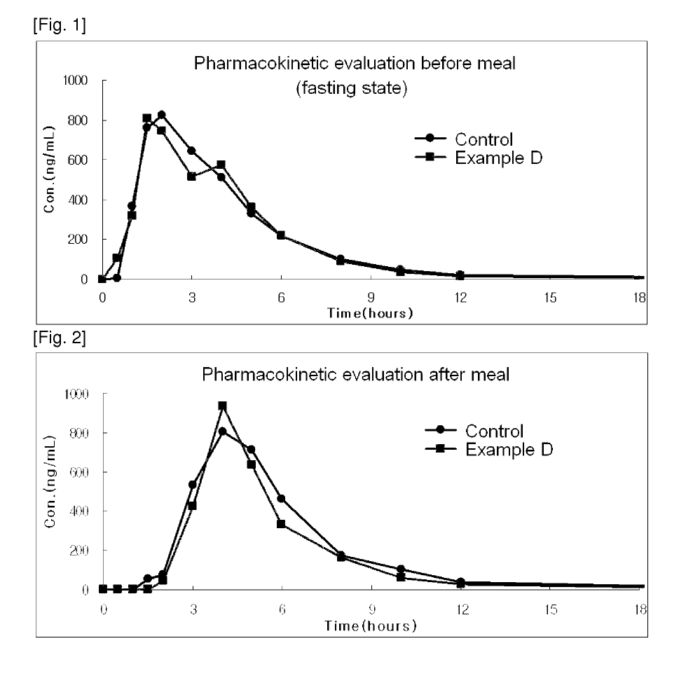

In capsules, example D using 710-600 um (diameter) of spherical sugar as seed showed Cmax and AUC more similar to the comparative example (commercially available product) than example C using 850-710 um (diameter) of spherical sugar as seed. When compared to the comparative example (commercially available product), example C showed 87.9% of Cmax and 106.6% of AUC, and example D showed 108.4% of Cmax and 96.1% of AUC. Result of example D was shown in FIG. 1.

To evaluate pharmacokinetic absorption after a meal, pharmacokinetic absorption patterns of the comparative example and example D were evaluated after High-fat breakfast (750 kcal, fat was about 50% of total calories (41.7 g)) was ingested. Results were shown in table 21 below and FIG. 2.

TABLE 21

| Batch no. | Example D | Comparative example |
|---|---|---|
| Cmax | 979.1 | 991.1 |
| AUC | 2914.7 | 3045.2 |
| Tmax | 4.1 | 4.4 |

In after-meal bioequivalent test to evaluate the effect of food, the example D having improved stability according to the present invention showed similar absorption ratio to the comparative example (commercially available product). Example D showed 98.8% of Cmax and 95.7% of AUC compared to the comparative example.

In the pharmacokinetic absorption test, LC/MS/MS was used for detecting esomeprazole in plasma, and sildenafil was used as internal standard. LC conditions were like “Column—YMC Hydrosphere C18; Mobile phase—10 mM Ammonium Acetate:Acetonitrile=10:90 (v/v); Flow rate—0.25 mL/min; Injection volume—10 uL; Sample temperature -10° C.; and column temperature—30° C.”, and MS conditions were like “Ionizing method—electrospray ionization (ESI) in Positive ion mode; MRM (Multiple Reaction Monitoring)—Esomeprazole [M+H]⁺ 326.2→198.1, Sildenafil [M+H]⁺ 475.2→283.2.”

1. A solid dispersion comprising esomeprazole free base or its alkali salt; alkaline material; and hydrophilic polymer selected from the group consisting of polyvinylpyrrolidone, vinylpyrrolidone-vinylacetate copolymer and their mixture.

2. The solid dispersion of claim 1, wherein the alkaline material is MgO or Mg(OH)₂.

3. Esomeprazole-containing pharmaceutical composition having improved stability, comprising the solid dispersion of claim 1.

4. A method for manufacturing esomeprazole-containing pharmaceutical composition having improved stability, comprising
(S1) preparing solution or suspension comprising esomeprazole free base or its alkali salt; magnesium oxide or magnesium hydroxide; and polyvinylpyrrolidone, vinylpyrrolidone-vinylacetate copolymer or their mixture; and
(S2) drying the solution or suspension.

5. The method of claim 4, wherein the drying of (S2) step is performed until LOD (loss on drying, 105° C., test sample weight 5 g) is less than 2%.
